(19) United States
(12) Patent Application Publication
(10) Pub. No.: US 2026/0051380 A1
(43) Pub. Date: Feb. 19, 2026

(54) AI-ENABLED DIGITAL PLATFORM FOR ACCELERATING SUBJECT ACCESS TO ADVANCED THERAPY MEDICINAL PRODUCTS

(71) Applicant: Genoplex, Inc., Boulder, CO (US)

(72) Inventors: Shoukri Kattan, Toronto (CA); Christopher Leidli, Boulder, CO (US)

(21) Appl. No.: 19/298,890

(22) Filed: Aug. 13, 2025

Related U.S. Application Data
(60) Provisional application No. 63/683,101, filed on Aug. 14, 2024.

(51) Int. Cl. G16H 20/00; G06F 40/205; G16H 50/20; G16H 50/70

(52) U.S. Cl. CPC G16H 20/00; G06F 40/205; G16H 50/20; G16H 50/70

(57) ABSTRACT

A computer implemented method for matching subjects with a treatment center. The method may comprise receiving a set of clinical data of a subject from a user. The method may comprise processing the set of data based at least in part on a computer program configured to identify and output treatment information. The method may comprise identifying, through an algorithm, a list of treatment centers based at least in part on the set of information. The method may comprise providing an output identifying relevant treatment centers.

### CROSS-REFERENCE TO RELATED APPLICATIONS

This application claims the benefit of U.S. Provisional Application No. 63/683,101, filed Aug. 14, 2024, which is incorporated by reference herein in its entirety.

### BACKGROUND

Gene cell therapy/advanced therapy medicinal products (AMTPs) rely on tissue engineering techniques to target underlying causes of diseases such as genetic changes. Development of these and other therapies is costly and time consuming, and may often requiring many years to undergo clinical trials and receive FDA approval. Once approved, not all AMTPs and gene cell therapies are offered at all treatment centers. Finding a treatment center that offers an AMTP relevant for a given disease of a subject is itself complex and time consuming, which may lead to subjects giving up. The unnecessary complexity of the current system, during the clinical trial phase and after FDA approval leads to late or missed treatment in subjects with disease where those subject may experience improved prognosis when treated earlier. This may increase economic strain on families, friends and the economy as a whole. Lower enrollment in clinical trials leads to manufacturers losing valuable resources to spoilage or needing to extend the length of their trials until they have sufficient data for FDA approval. Improving the process of discovering a treatment center with the appropriate clinical trials or FDA approved AMTPs is essential to improving the efficacy of the health care system overall, especially as more advanced treatments with higher development costs become the norm in treating diseases of all kinds.

### SUMMARY

In one aspect, the present disclosure provides a computer-implemented method to match subjects with a treatment center comprising, via a computer system, comprising: (a) receiving a set of clinical data of a subject from a user; (b) processing the set of data based at least in part on a computer program configured to identify and output treatment information; (c) identifying, through an algorithm, a list of treatment centers based at least in part on the set of information; and (d) providing an output identifying relevant treatment centers.

In some cases, the set of data comprises at least one of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, a measure of subject prognosis, or any combination thereof. In some cases, the clinical code comprises an ICD10 code. In some cases, clinical data comprise HIPAA-limited data. In some cases, the clinical data is fully identified. In some cases, the clinical data is vectorized. In some cases, the clinical data is encrypted. In some cases, the encryption is two-way. In some cases, the algorithm comprises a rule-based system. In some cases, the rule-based system parses at least an FDA approved indication of clinical trial.

In some cases, the rule-based system comprises (a) a rule for minimizing distance between a subject and a treatment center; and (b) a rule for matching a set of treatment centers with the subject. In some cases, the rule of (a) is based on a distance threshold. In some cases, the distance threshold is tunable. In some cases, the method further comprising delivering the user a list of financial assistance options. In some cases, the method further comprising providing transportation options. In some cases, the set of treatment centers houses a clinical trial that matches with at least a portion of the set of information relevant to a treatment. In some cases, the clinical trial comprises a cellular therapy clinical trial. In some cases, the clinical trial comprises a gene therapy clinical trial. In some cases, the clinical trial comprises a radioligand therapy clinical trial. In some cases, the clinical trial comprises a Tissue Engineered Product clinical trial. In some cases, the cellular therapy is a Somatic Cell Therapy Medicinal Product. In some cases, the computer program is a machine learning model. In some cases, the machine learning model comprises a neural network. In some cases, the machine learning model comprises a large language model.

In some cases, the identifying the list of treatment centers comprises: (a) parsing, through the algorithm, an FDA-approved indication associated with a treatment; (b) associating the set of information with the parsed FDA-approved indication; and (c) pulling the list of treatment centers where the treatment is performed. In some cases, the algorithm comprises a machine learning model. In some cases, the machine learning model comprises an autoencoder. In some cases, the machine learning model comprises a long short-term memory model. In some cases, the machine learning model comprises a large language model. In some cases, the machine learning model comprises a recurrent neural network. In some cases, the machine learning model comprises a clustering algorithm. In some cases, the algorithm comprises a transformer. In some cases, the neural network comprises a transformer. In some cases, the neural network comprises an autoencoder. In some cases, the listing of relevant treatment centers is arranged in order of distance to the subject. In some cases, the listing of relevant treatment centers comprises at least one of a URL, an address, a phone number, a contact person, a physical mailing address, an email address, instruction regarding enrollment, accepted insurance, provider names, an estimation of distance from the subject's approximate location, a list of nearby accommodations, a hyperlink, or any combination thereof. In some cases, the user and the subject are the same person. In some cases, the user and the subject are different people. In some cases, the user is a licensed medical provider. In some cases, the clinical data stored on a cloud-based system. In some cases, some combination of the computer program, the algorithm, or the transforming is performed on a cloud-based system. In some cases, the clinical code comprises an ICD-11 code. In some cases, the clinical code comprises a SNOMED CT code. In some cases, the clinical code comprises a LOINC code. In some cases, the clinical code comprises a CPT code. In some cases, the clinical code comprises a HCPCS code. In some cases, the clinical code comprises an ICD-O code. In some cases, the clinical code comprises a DRG code. In some cases, clinical code comprises a Read Codes. In some cases, clinical code comprises an RxNorm code. In some cases, clinical code comprises an ATC code. In some cases, the rule-based system parses at least an FDA approved AMTP. In some cases, the rule-based system parses at least an FDA approved therapy. In some cases, the set of data comprises any two members selected from the group consisting of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis. In some cases, the set of data comprises 5 members selected from the group consisting of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis. In some cases, the set of data comprises any 10 members from the group consisting of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis. In some cases, the set of data comprises subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis. In some cases, the set of data comprises all of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis.

The present disclosure provides a non-transitory computer-readable medium comprising machine executable code that, upon execution by one or more computer processors, implements any of the methods above or elsewhere herein.

Another aspect of the present disclosure is a system comprising one or more computer processors and computer memory with machine executable code that, upon execution by the one or more computer processors, implements any of the methods above or elsewhere herein.

Existing clinical trial databases and systems can be web-based resources that provide subjects, providers, physicians, researchers, and the general public with access to information about publicly and privately supported clinical studies. Often, there are a large number of clinical trials being conducted at any given time for many indications and with many types of treatments being tested (such as, but not limited to, cell therapy, gene therapy, radioligand therapy, tissue-engineered product (TEP) therapy, and somatic cell therapy medicinal product (sCTMP)). The clinical trials may relate to a wide range of diseases and conditions. In some instances, clinical trials are performed at or using the resources of multiple sites, such as hospitals, laboratories, and universities.

Clinical trial databases and systems may receive information on each clinical trial via the data submission by the principal investigator (PI) or sponsor (or related staff). For example, the public website clinicaltrials.gov is maintained by the National Library of Medicine (NLM) at the National Institutes of Health (NIH). Most of the records on clinicaltrials.gov describe clinical trials.

The information on clinicaltrials.gov is typically provided and updated by the sponsor (or PI) of the particular clinical trial. Studies and clinical trials are generally submitted (registered) to relevant websites and databases when they begin, and the information may be updated as needed throughout the study or trial. Studies and clinical trials listed in the database span the United States and over two hundred additional countries. Notably, clinicaltrials.gov and other clinical trial databases may not contain information about all the clinical trials conducted in the United States (or globally) because not all studies are currently required by law to be registered. Additionally, trial databases are often not maintained to include the most up-to-date information about the conduct of any particular study.

In general, each clinical trial record (such as on clinicaltrials.gov) presents summary information about a study protocol, which can include the disease or condition, the proposed intervention (e.g., the medical product, behavior, or procedure being studied), title, description, and design of the trial, requirements for participation (eligibility criteria), locations where the trial is being conducted (sites), and contact information for the sites.

Notably, clinical trial databases and websites may express clinical trial information using free text (i.e., unstructured data). Inclusions may describe characteristics that a prospective subject may have if they are to be included in the study. Exclusions may generally describe characteristics that disqualify prospective subjects from inclusion in the study. Inclusion and exclusion criteria may comprise factors such as age, gender, race, ethnicity, type and stage of disease, the subject's previous treatment history, and the presence or absence (as in the case of the “healthy” or “control” subject) of other medical, psychosocial, or emotional conditions.

When described with free text, inclusion criteria may require a physician or other person to review the inclusion criteria compared to a subject's medical record to determine whether the subject is eligible for the study. Some subject health information is structured data, where health information resides within a fixed field within a record or file, such as a database or a spreadsheet. The free-text nature of the inclusion criteria presented by websites such as clinicaltrials.gov may not lend itself to simple matching with structured data. The inclusion criteria described on the website require analysis of multiple structured data fields. The challenges posed by free text descriptions of clinical trials make rapid identification of clinical trials burdensome, making matching subjects to clinical trials unnecessarily complex. Finding a clinical trial may be left up to the subject or a family member who may have difficulties understanding the descriptions, inclusions, and exclusions. Healthcare professionals may be better suited to finding a clinical trial. Still, the task is often time-consuming, requiring searching databases, reading trial language, and collating findings to surface suitable trials. In some cases, a search may be incomplete. Subjects who could enroll in a clinical trial may not discover that a suitable trial exists and never have the opportunity to enroll.

Compounding this issue is difficulty finding a treatment center for a subject enrolled in a clinical trial. A challenge with finding a treatment center for a subject is often the subject's proximity to a treatment center with an appropriate clinical trial. Enrollment, in a clinical trial is often routed through treatment centers. Each treatment center has its own enrollment process which must be navigated. Many treatment centers subject intake/enrollment process includes a manual process such as a faxed form, or enrollment by telephone causing the process to be slower. The difficulty of find an appropriate clinical trial, finding a treatment center, and navigating one or more treatment centers enrollment process is especially and unnecessarily onerous. Additionally, manufacturers of therapies have difficulty in knowing how much of a therapy to produce for a trial, and how much and where to stock the produced therapies. The issue of subject enrollment challenges along with manufacturer logistical challenges can result in many subjects who would be good candidates for a trial being left unenrolled and high costs to spoilage of therapies when they are misallocated.

Thus, there is a long-felt need for a method and system for connecting subjects with a treatment center for a clinical trial. The method may comprise the use of a corresponding system. Sometimes, the subjects may be connected to a treatment center and enrolled within a seamless graphical user interface. An advantage of such an interface may be removing an additional barrier of finding enrolment pages or documents for different clinical trials across different treatment centers. In addition, the systems, methods and models described herein may improve the number of people participating in a clinical trial. In turn, higher enrollment levels in clinical trials may improve a trial's clinical data and outcomes. Healthcare professionals may be better able to serve their subjects by efficiently identifying relevant clinical trials. The systems, methods and models described herein may improve health equity for subjects residing in remote areas by offering them or their primary healthcare professionals an improved tool to aid in their decision to enroll in a clinical trial.

### INCORPORATION BY REFERENCE

All publications, patents, and patent applications mentioned in this specification are herein incorporated by reference to the same extent as if each publication, patent, or patent application was specifically and individually indicated to be incorporated by reference. To the extent publications, patents, and patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings (also “Figure” and “FIG.”herein), of which:

FIG. 1 shows a non-limiting example of a system for matching subjects with a treatment center in accordance with some embodiments described herein.

FIG. 2 shows a non-limiting example of a computing device; in this case, a device with one or more processors, memory, storage, and a network interface in accordance with some embodiments.

FIG. 3 shows a non-limiting example of a web/mobile application provision system; in this case, a system providing browser-based and/or native mobile user interfaces in accordance with some embodiments.

FIG. 4 shows a non-limiting example of a cloud-based web/mobile application provision system; in this case, a system comprising an elastically load balanced, auto-scaling web server and application server resources as well synchronously replicated databases in accordance with some embodiments.

FIG. 5 shows a non-limiting example of a representational state transfer (REST) application programming interface (API) and various web services that may be carried out.

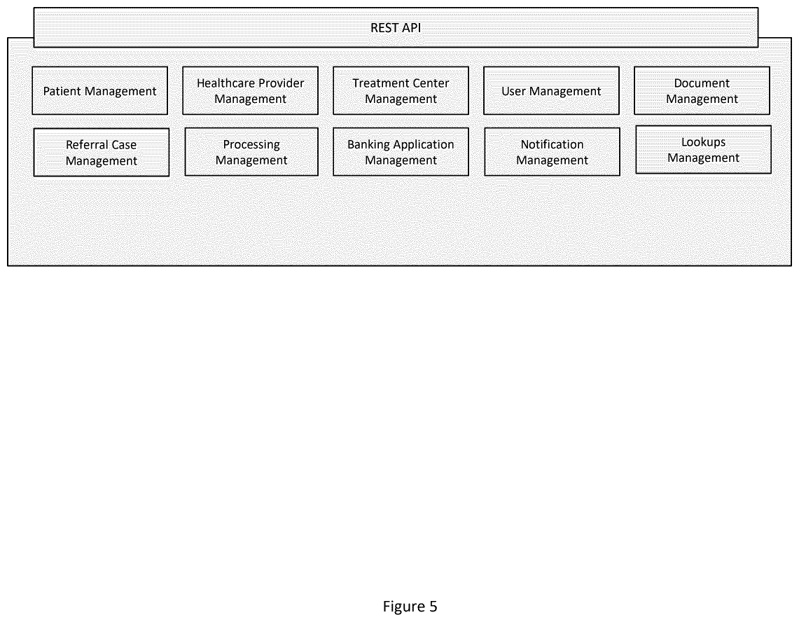

FIG. 6 shows a schematic of various platform and API interfaces that may interact with the methods disclosed herein.

### DETAILED DESCRIPTION

The systems, methods, and models described herein may be implemented by a computer system and may be used to enhance matching of a subject with one or more treatment centers. The systems, methods and models described herein may be implemented by a computer system and may be used to enhance matching of a subject with one or more treatment centers and treatment providers. The systems, methods and models described herein may be implemented by a computer system and may be used to enhance matching of a subject with one or more treatment centers and therapeutic options. The systems, methods and models described herein may be implemented by a computer system and may be used to enhance matching of a subject with one or more clinical trials. The systems, methods and models described herein may be implemented by a computer system and may be used to enhance matching of a subject with one or more clinical trials and treatment centers.

The systems, methods and models may be used for recommending and connecting a subject to a treatment center and/or clinical trial. The systems, methods and models may enhance the speed of access to enrollment in the treatment center and clinical trial.

The disclosure includes systems, methods and models that may track a subject's progress from enrollment into therapy and/or through the completion of the clinical trial.

A system may be configured to implement a method of the disclosure. A model may be implemented by a system as part of a method. The system may provide information to the manufacturer of a therapy, treatment, or medicine. The methods and systems may serve as a communication platform. The methods and systems may be configured to collect real-time data.

The disclosure includes developing or training a machine learning model to match subjects to clinical trials. Methods of the of the disclosure may include using the trained machine model to match subjects to clinical trials. Systems of the disclosure may be programmed and used to execute the machine learning model to match subjects to clinical trials.

The systems, methods and models may be used to forecast manufacturer needs. The systems, methods and models may provide recommendations for financial assistance to subjects. The systems, methods and models may provide recommendations for transport options for subjects. Details of these and other embodiments may be found herein.

### Data and Data Transformation

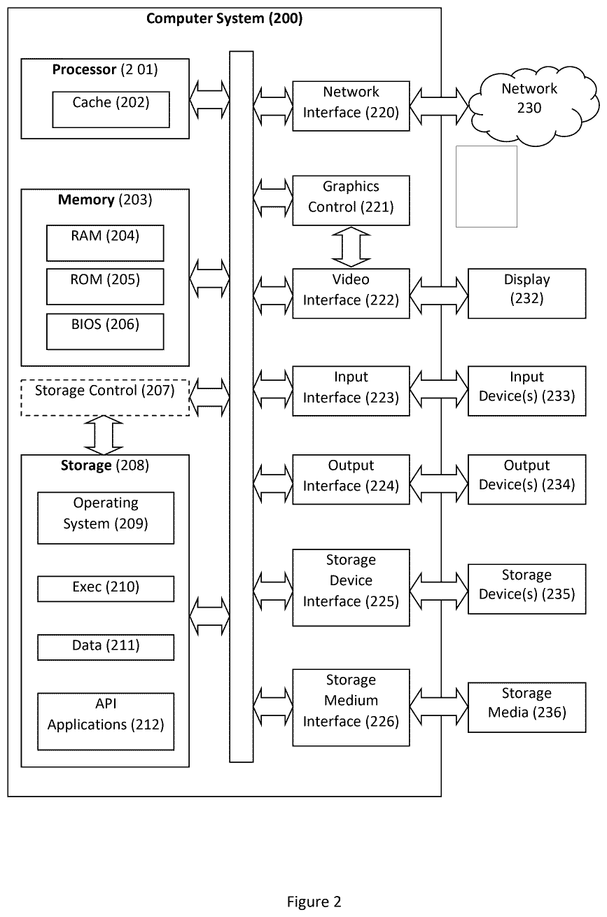

The systems, methods and models disclosed herein may make use of data. The data may include clinical data. The systems, methods and models disclosed herein receive and transform clinical data using a computer system such as that depicted in FIG. 2. Clinical data and/or transformed clinical data may be used to train a model that implements any one or more of the method steps as described herein. For example, the model may match a subject with a clinical trial.

Clinical data may, for example, include demographic data, medical history data, diagnostic data, health outcomes data, referral and treatment center data, health survey data, genomic and biomarker data, efficacy and safety data, HIPAA-related data, and clinical codes.

Clinical data may include subject demographic data. Examples of demographic data include age, date of birth, gender, race, ethnicity, income, name, known allergies, vaccination history, vaccination status, occupation, address, contact details, insurance information, medical history, treatment history, medication history, current medications, past medications, past treatment history, drug usage, and familial medical history. In some embodiments, subject demographic data may be tabular.

The clinical data may include diagnosis data. Examples of diagnostic data include results of lab tests, scans, an indication of a disease, an indication of a condition, results of a molecular test, results of a genotyping panel, results of a whole genome sequencing, blood pressure, heart rate, blood glucose level, cholesterol level, pulmonary function, PET scan, MRI scan, Papanicolaou (Pap) test, results of a blood test, result of an eye test, result of cancer screening, qrtPCR, PCR, mammography, laparoscopy, urine analysis, fluoroscopy, enzyme-linked immunosorbent assay (ELSIA), endoscopy, endoscopic retrograde cholangiopancreatography (ERCP), electrophysiologic testing, electromyography, electroencephalograph (EEG), electrocardiogramdiography, x-ray, culture, cone biopsy, colonoscopy, colposcopy, computed tomography (CT), chromosomal analysis, chronic villus sampling, bronchoscopy, bone marrow aspiration, barium x-ray, auscultation, audiometry, tympanometry, thoracoscopy, thoracentesis, spirometry, genomic data, biomarker data, efficacy data, safety data, safety and/or efficacy data required for meeting regulatory requirements, safety and/or efficacy data required for meeting regulatory requirements for up to 15 year safety follow up for gene therapy.

The clinical data may include referral, provider and treatment center data. The clinical data may include referral location. The clinical data may include the referral date. The clinical data may include inbound treatment center contact information. The clinical data may include treatment center capacity. The clinical data may include treatment center turn-around time. The clinical data may include treatment center operational data.

The clinical data may include health outcomes data. The health outcomes data may include subject reporting outcomes (PRO) data, time-based data (such as, but not limited to, time from referral to treatment). The clinical data may include a measure of subject prognosis.

The clinical data may include subject history data. The clinical data may include familial history data. The clinical data may include medical history data. The clinical data may include lab result data. The clinical data may include health survey data.

The clinical data may include a variety of other types of data. The clinical data may include ICEES data. The clinical data may include Columbia open health data (COHD). The clinical data may include HIPAA Safe Harbor (HuSH) data. The clinical data may include HIPAA Safe Harbor Plus (HuSH+) clinical data. The clinical data may include HIPAA-limited. The clinical data may include fully identified data. In some embodiments clinical data may comprise a clinical code. In some embodiments a clinical code may be an icd10 code. In some embodiments a clinical code may be an CPT code. In some embodiments data may be encrypted. In some embodiments data may be two way encrypted, in some embodiments data may be one way encrypted.

The data may be transformed. The transformation may be performed by an algorithm. The algorithm may comprise a text parsing algorithm. The transformation may be a diffeomorphic method. The transformation may be symmetrical diffeomorphic elastic matching. The transformation may be a translation, or a rigid transformation (such as an Euler transform), an isotropic scaling, an affine transformation, a spline-based transformation (such as a b-spline to thin plate spline). The algorithm may be a machine learning model. The machine learning model may comprise a neural network. The neural network may comprise an attention network. The neural network may comprise a transformer. The neural network may comprise an autoencoder. The neural network may comprise an encoder. The neural network may comprise a decoder. The neural network may comprise a convolutional neural network (CNN). The CNN may include 3d convolution, i.e., volumetric convolution such as on MRI or CT scan, or spatiotemporal feature extraction from video data. The neural network may comprise a recurrent neural network (RNN). The neural network may comprise a long, short term memory (LSTM) model. The neural network may comprise a gated recurrent unit (GRU). Neural networks such as RNN, LSTM, GRU and attention networks may be used to detect spatiotemporal dependencies, contextual information, longitudinal (such as time based) dependencies and/or combination thereof. The neural network may comprise a graph neural network (GNN) The neural network may comprise a u-net. The neural network may comprise skip connections. The neural network may comprise an image segmentation model.

In some embodiments a video analysis model may be used. The video analysis may include an optical Flow Algorithms. The video analysis may include a spatiotemporal attention mechanism. The video analysis may include an action recognition model. The video analysis may include a video segmentation model. The video analysis may include a temporal convolutional network (TCN). The video analysis may include a video anomaly detection method such as an unsupervised or semi-supervised learning to identify unusual events in medical videos.

### Recommender System

In some embodiments the methods and systems disclosed herein may include a recommender system, as discussed herein. The recommender system may include content-based filtering. Content based filtering may be used to recommend clinical trials or treatment centers based on subject characteristics. The recommender system may include collaborative filtering. Collaborative filtering may be used to find and/or leverage similarities between subjects and clinical trials and/or treatment centers to find and/or recommend at least one treatment center or trial to a user.

The methods and systems disclosed herein may compile clinical data information into a database. The methods and systems may transform at least one of the descriptions of a clinical trial, the inclusions of a clinical trial, the exclusions of a clinical trial, or some combination thereof. The description, inclusions, and/or exclusions may be transformed.

The clinical trial description, clinical trial inclusions, and/or clinical trial exclusions may be transformed. The transformation may be performed by an algorithm. The algorithm may comprise a text parsing algorithm. The algorithm may be a machine learning model. The machine learning model may comprise a neural network. The neural network may comprise an attention network. The neural network may comprise a transformer. The neural network may comprise an autoencoder. The neural network may comprise an encoder. The neural network may comprise a decoder. The neural network may comprise a convolutional neural network (CNN). The neural network may comprise a recurrent neural network (RNN). The neural network may comprise an long, short term memory (LSTM) model. The neural network may comprise a u-net. The neural network may comprise skip connections. The neural network may comprise an image segmentation model. The neural network may comprise a large language model (LLM).

The clinical data and the clinical trial description, inclusions, and/or exclusions may be processed by the same model. The model produces a score to rank similarity between at least one part of the clinical data and at least one member selected from the group consisting of a clinical trial description, clinical trial inclusions, and clinical trial exclusions. The score may be a distance metric based at least in part on the latent space of the model. The distance may be calculated by Euclidian distance. The distance may be calculated by Manhattan distance. The distance may be calculated by Minkowski distance. The distance may be calculated by hamming distance. The distance may be calculated by cosine distance.

The clinical trial description, inclusions and/or exclusions may be parsed. The parsed clinical trial description, inclusions and/or exclusions may be matched to a subject. In some embodiments a set of treatment centers for the matched clinical may be compiled. The set of treatment centers may be ranked using a rule-based system. The rule-based system may minimize distance. The distance may be from the address of the subject to the treatment center being ranked. The set of treatment centers may be trimmed according to a distance threshold where any treatment center further from the subject's address may be excluded from the set of treatment centers. The threshold may be selectable by the user.

Pursuant to methods and systems, a set of (1 or more) clinical trials may be identified based on the score from the model. The methods and systems may query a list of treatment centers associated with the set of clinical trials. The methods and systems may provide an output identifying the treatment centers associated with the set of clinical trials. The identification may include an address, a center name, a physician name, a physician contact, and/or center contact information.

The methods and systems provide an option to enroll in a clinical trial at a treatment center. The methods and systems may require an indication of the user's identity as a health care provider or a subject. The methods and systems may display different enrollment screens based on the identification of the user as a healthcare provider or a subject.

The user may be a subject. The user may be a health care provider. The user may be a legal representative of the subject. The user may be a legal guardian of the subject.

The methods and systems may provide communications to a user after enrolment in a clinical trial at a treatment center. The methods and systems may provide information about a manufacturer's therapy to the manufacturer. The methods and systems may provide communications about the clinical trial with the treatment center. The methods and systems may provide communications about the clinical trial with individuals involved in conducting the clinical trial. The methods and systems may provide communications to the health care provider about the subject and/or clinical trial.

### Workflow Automation

In some embodiments the methods and systems disclosed herein may include workflow automation, as discussed herein. A workflow automation method may comprise an agentic AI. An example of a workflow automation may utilize methods of data transformation. A workflow automation may utilize one or more machine learning models. Workflow automation may utilize a vectorization of data which may be the result of a data transformation. The vectorization may be used as input to a workflow automation method. The workflow automation method may be an artificially intelligent method. The workflow automation method may comprise a machine learning model. The workflow automation method may comprise a plurality of machine learning models, wherein each of the plurality of machine learning models may be communicatively coupled. A workflow automation method may work in real-time or near real-time (e.g., real time with a sufficient delay to perform computations and processing or communications among modules, or various members of a network). An example of a workflow automation method may be an AI orchestration engine which may coordinate tasks in real time.

The workflow automation method may be configured to act towards a goal. A goal may be communicated to the workflow automation system by a user. For example, a goal may be typed into an interface with an agentic AI wherein the goal is processed by a large language model to transform the goal into actionable information. Examples of actionable information may be a vectorization of the user provided goal, a language output from an LLM that breaks the goal into portions, a vectorization of portions of the user input goal, a set of vectorizations of each portion of a set of portions. A goal may be provided as a hardcoded or API-defined goal. For example, a goal may be passed via a function call, API or system configuration. A goal may be determined based on context. For example, a goal may be implied via a success condition, world state, reward, or any combination thereof. A success condition may be a condition for which, when reached, indicates a task and/or workflow is completed.

A workflow automation method may be configured to analyze electronic health data (EHR) data (e.g., EMR data). Examples of EHR data may include structured data (such as ICD, CPT and/or lab results) and/or unstructured data (such as chart data, clinical notes, and/or radiology reports). Other non-limiting examples of EHR data may include clinical notes, lab notes, biomarker and diagnosis codes. In some cases EHR data may be analyzed to identify patients who meet a clinical criteria for one or more diseases. Analysis of EHR data may include detecting patterns in data that may be indicative of a disease. A non-limiting example of a disease may be a rare disease.

Upon detection of a pattern that may be indicative of disease a workflow automation method may identify a subject (such as a patient) as eligible for a treatment (such as a personalized medicine and/or advanced therapy). A workflow automation method may match a subject to a treatment center (as described herein). Subsequent to matching a treatment center a workflow automation method may perform digital intake workflows. A digital intake workflow may include, for example, capturing consent, recording insurance information, assisting in filling out pre-authorization documents, and/or routing a subject to a treatment center.

A workflow automation method may facilitate, assist in or perform coordination of patient (e.g., subject) care. A workflow automation method may assist a user (such as a service line coordinator) in clinical and/or financial workflows. Non-limiting examples of clinical and/or financial workflows may include form completion, and/or patient communication.

Non-limiting examples of workflow automation may include top-of-funnel marketing support (such as for treatment centers to market the therapies provided and expertise in disease treatments). Patient intake AI, physician-companion AI, integration into EHR environments, integration into practice management software, a revenue cycle management suite, intake, billing doc intel or any combination thereof.

In some cases, the workflow automation method may be an end-to-end automation solution which may comprise patient identification, intake, and treatment center matching. In some cases the workflow automation method may be a plug-and play method which may integrate into provider environments (e.g. community, hospital, academic, or integrated delivery network environments) without altering those environments existing systems. In some cases the plug-and-play capability may be carried out through an application programming interface (API). For example, an API may facilitate the capabilities of the workflow automation method described herein. This may be achieved through standardized protocols (e.g., REST, GraphQL, etc.), SDK's or client libraries which may handle authentication, request building, error parsing and or retry logic all of which may interface with the provider environment with minimal or no setup. Integration may be done in a manner that enables quick and minimal testing and integration (such as through Postman collections or OpenAPI which may describe the methods functionality. Updates or changes to the method may be managed using events, hooks, or webhooks which notify the provider environment of updates or changed or may subscribe to events (such as a new user creation, treatment center matching, form completion, form upload, form download, new communication from patients or provider, etc.) without polling the API. The API may comprise an adapter that may be between the provider environment and the method to perform adaptive or middle processing tasks such as data mapping, error handling, logging, translation of api calls into formats suitable to the provider environments processing or translation of the provider environments formats to those understandable by the API, etc. The API may comprise auto-discovery of services or endpoints such that the method may detect parts of the provider environments (such as existing software's, and/or databases with which the method may communicate or be integrated with) and allowing the method to configure portions of its communications based on those services or endpoints automatically to ease setup time.

Non-limiting examples of formats which the API may communicate with or be configured to translate may include Fast Healthcare Interoperability Resources (FHIR), Health level seven (HL7), PDF, plain-text or any combination thereof.

In some embodiments, the methods and systems provided herein may comprise a Billing Agent. A billing agent may be autonomous. A billing agent may determine an appropriate payer workflow. A billing agent may selects codes. A billing agent may build the submission package. A billing agent may file a submission package electronically. A billing agent may tracks responses. Advantageously, such a method may eliminate manual RCM tasks making more efficient the billing cycle and/or negotiations. For high-cost therapies this may improve both the time to therapy and the overall administrative cost of the therapy both improving patient outcomes and workflow efficiency while delivering potentially live saving or life changing therapy at lower cost and faster.

In some embodiments a billing agent may determine, from patient-specific data and insurer policy data, a prior-authorization workflow. Examples of such codes for a workflow my include, but aren't limited to CPT, HCPCS, ICD-10-CM, or ICD-10-PCS, or any combination thereof. In some embodiments a billing agent may generate an electronic submission package containing the selected codes and supporting clinical evidence. In some embodiments, a billing agent may transmit the electronic submission package to a payer computer interface. In some embodiments, a billing agent may receive via polling or webhook, a response message indicating approval, denial, or request for additional information. In some embodiments a billing agent may compute a probability-of-approval score and, if below a threshold, an alternate package may be auto-generated. As such the billing agent may predict the probability of an approval based on the current package and responsive to the probability being below a set threshold may generate a new package. The prediction of a probability of approval and re generation of a new package may repeat until the predicted probability of approval is above a given threshold. A threshold may be greater than 30%, greater than 40%, greater than 50%, greater than 60%, greater than 70%, greater than 80%, greater than 85%, greater than 90%, greater than 91%, greater than 92%, greater than 93%, greater than 94%, greater than 95%, greater than 96%, greater than 97%, greater than 98%, greater than 99%. In some embodiments, code selection may leverage a large language model (LLM) fine-tuned on historical claim outcomes.

In some embodiments, A workflow automation method may comprise multiple workflow automation agents. Different workflow automation agents may perform different tasks and/or context stores (e.g., unstructured health and or EMR information used in the clinical or financial workup of a consumer or patient). For example, separate agents may perform intake billing and doc intel related tasks. In some embodiments, a meta-agent may monitor outcomes of the workflow automation method as a whole and may fine-tune individual agents based on overall method outcomes to improve performance. In some embodiments the workflow automation method may comprise an orchestration hub may be configured to register a plurality of autonomous software agents.

In some embodiments the workflow automation method may comprise a shared context data store operatively coupled to the orchestration hub. A shared context data store may be a mechanism or area where data can be stored and accessed by multiple components, applications, or parts of a system, facilitating communication and data sharing between them (such as Redis, Amazon DynamoDB, MongoDB, Apache Cassandra, Graph databases, Elasticsearch, SQL, or timeseries databases). A context store may comprise a distributed or centralized data storage method and/or system.

In some embodiments the methods and systems provided herein may comprise a treatment plan reimbursement optimizer (TPRO). In some embodiments, the TRPO may generate a likelihood that a proposed treatment regimen will be reimbursed. In some embodiments the likelihood of reimbursement may be compared against a threshold. and, if the score is low (such as lower than a threshold), the TRPO may produce an alternate plan (e.g., a treatment plan that meets clinical guidelines). In some embodiments the TRPO may assess whether the likelihood of approval is increased to be approved. In some embodiments the alternative treatment plan may be compared against a threshold. In some embodiments a treatment plan may be provided to the user for sending or sent by the workflow automation method to a provider or insurance provider responsive to a likelihood of approval being above a threshold or higher than a previous likelihood of a earlier treatment plan.

In some embodiments, the methods and systems provided herein may comprise a method for optimizing a treatment plan. The method for optimizing a treatment plan may receive a proposed treatment plan comprising at least one therapeutic regimen.

The proposed treatment plan may be provided by a user. The proposed treatment plan may be generated by a machine learning model. The method for optimizing a treatment plan may access payer policy data and historical claim data. The method for optimizing a treatment plan may calculate a likelihood-of-reimbursement metric for the proposed treatment plan. The method for optimizing a treatment plan may, when the metric is below a predefined threshold, automatically generate an alternate treatment plan (e.g., a treatment plan compliant with clinical guidelines) and exceeding the threshold. A threshold may be greater than 30%, greater than 40%, greater than 50%, greater than 60%, greater than 70%, greater than 80%, greater than 85%, greater than 90%, greater than 91%, greater than 92%, greater than 93%, greater than 94%, greater than 95%, greater than 96%, greater than 97%, greater than 98%, greater than 99%. In some embodiments, code selection may leverage a large language model (LLM) fine-tuned on historical claim outcomes.

In some embodiments he likelihood-of-reimbursement metric is produced by a machine learning model. Examples of a machine learning model may include, but are not limited to, a gradient-boosted decision-tree model, a neural network, a logistic regression, a linear regression, a support vector machine, a Bayesian model, a monte carlos model, a random forest, a decision tree, a latent Dirichlet model or any combination thereof. A machine learning model may trained on prior claim decisions. Such training may comprise using as input a treatment plan and using as labels a reimbursement decision. Such training may comprise using as input at least a portion of clinical data, clinical codes and/or diagnostic data. In such training a machine learning method may be optimized (e.g., using optimization techniques) to predict reimbursement decisions and/or probabilities of reimbursement decisions. In some embodiments additional generated treatment plans may be used to train a model (such as a treatment optimization method) to generate treatment plans with improved reimbursement likelihoods. Such training may comprise adversarial training, and or reinforcement learning. Adversarial training may comprise generating a treatment plan through a machine learning model, inputting the treatment plan into a machine learning model configured to estimate a likelihood of reimbursement. Responsive to said input the machine learning model configured to estimate a likelihood of reimbursement ma output a likelihood of reimbursement. Such input and output may occur over a plurality of generated treatment plans. The machine learning model that generated the treatment plans may then be optimized based on the output of the machine learning model configured to estimate a likelihood of reimbursement such that it may output improved treatment plans (e.g., more likely to be reimbursed or approved). Such methods may also be used with approval and/or reimbursement decision from a insurance provider such (e.g., the insurance provider decision in used for optimization of the treatment plan generating machine learning model). In some embodiments input to the treatment plan generating machine learning model may comprise an indications of a specific insurance provider. In some embodiments the machine learning model that generates the treatment plan may be fine tuned for specific insurance providers.

Depending on a patient's medical policy and payer coverage plan, a patient may not be eligible for a particular treatment In some embodiments, the alternate treatment plan is presented with an explainability report citing payer rules triggered. In some embodiments, an Agentic AI model or a plurality of agentic AI models may assess patient documentation to support benefits verification. In some cases, the agentic AI model may determine that a particular treatment plan or portion of a treatment plan may not be covered and may request, suggest, and/or recommend an alternative treatment by the provider.

In some embodiments the workflow automation method may comprise an Intake Agent that receives patient-specific clinical data and writes the data to the shared context data store. In some embodiments the workflow automation method may comprise a Billing Agent that retrieves the patient-specific clinical data, generates a payer-specific prior-authorization request, and electronically transmits the request to a payer computer system. In some embodiments the workflow automation method may comprise a meta-agent configured to monitor outcome data from each autonomous agent and, based at least in part on the outcome data, update one or more parameters of the autonomous agents.

In some embodiments the workflow automation method may comprise a communications bus that facilitates message exchange between the orchestration hub and each autonomous agent. In some embodiments the workflow automation method may comprise a Document-Intelligence Agent using a vision-language transformer to extract structured entities from image-based documents. Examples of image based documents may include, but are not limited to, scans of documents (e.g., health data, lab results, genomics results, etc.) and medical scans (e.g., X-rays, CT scans, MRI scans, PET scans, ultrasound scans, DEXA scans, fluoroscopy scans, mammograms, SPECT scans, and nuclear medicine scans). In some embodiments the orchestration hub routes tasks according to a routing policy learned through reinforcement learning.

Advantageously, a workflow management method may provide a reduction in the time from diagnosis to the time to treatment. The methods and systems disclosed herein may facilitate the ease of often time-consuming steps in treatment center identification, intake paperwork and processing and patient communication which are often time consuming and stressful for patients, primary care physicians and families. Easing the burden on the individuals involved will allow patients and physicians to focus of timely and effective treatment of disease which may not only alleviate stress but may improve outcomes of treatment through earlier and more timely treatment. In addition, efficient processing may improve cost of patient intake and processing to a treatment center, ensuring all forms are completed correctly, verifying information in forms, and providing efficient workflows to speed up processes which can be time consuming. Also advantageously, a workflow automation method may identify traditionally hard to find patients with potentially rare or often misdiagnosed diseases. This method will lead to earlier treatment for many patients which will result in improved outcomes for patients and their families while easing the cost of treatment, stress of navigating the healthcare landscape (such as treatment center identification and form completion) and dealing with insurance which are all currently both time consuming, costly and stressful.

### Computer Systems

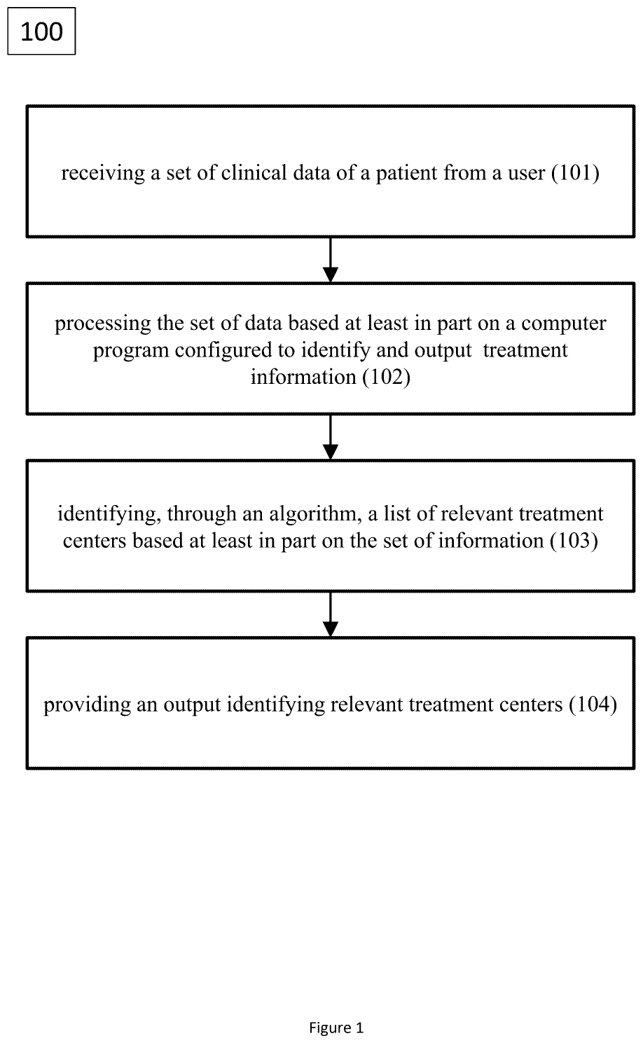

FIG. 1 depicts a non-limiting example of a computing system 100 for receiving a set of clinical data of a subject from a user 101, processing the set of data based at least in part on a computer program configured to identify and output treatment information 102, identifying, through an algorithm, a list of relevant treatment centers based at least in part on the set of information 103, providing an output identifying relevant treatment centers 104.

Referring to FIG. 2, a block diagram is shown depicting an exemplary machine that includes a computer system 200 (e.g., a processing or computing system) within which a set of instructions can execute for causing a device to perform or execute any one or more of the aspects and/or methodologies for static code scheduling of the present disclosure. The components in FIG. 2 are examples only and do not limit the scope of use or functionality of any hardware, software, embedded logic component, or a combination of two or more such components implementing particular embodiments.

Computer system 200 may include one or more processors 201, a memory 203, and a storage 208 that communicate with each other, and with other components, via a bus 240. The bus 240 may also link a display 232, one or more input devices 233 (which may, for example, include a keypad, a keyboard, a mouse, a stylus, etc.), one or more output devices 234, one or more storage devices 235, and various tangible storage media 236. All of these elements may interface directly or via one or more interfaces or adaptors to the bus 240. For instance, the various tangible storage media 236 can interface with the bus 240 via storage medium interface 226. Computer system 200 may have any suitable physical form, including but not limited to one or more integrated circuits (ICs), printed circuit, boards (PCBs), mobile handheld devices (such as mobile telephones or PDAs), laptop or notebook computers, distributed computer systems, computing grids, or servers.

Computer system 200 includes one or more processor(s) 201 (e.g., central processing units (CPUs), general purpose graphics processing units (GPGPUs), or quantum processing units (QPUs)) that carry out functions. Processor(s) 201 optionally contains a cache memory unit 202 for temporary local storage of instructions, data, or computer addresses. Processor(s) 201 are configured to assist in execution of computer readable instructions. Computer system 200 may provide functionality for the components depicted in FIG. 2 as a result of the processor(s) 201 executing non-transitory, processor-executable instructions embodied in one or more tangible computer-readable storage media, such as memory 203, storage 208, storage devices 235, and/or storage medium 236. The computer-readable media may store software that implements particular embodiments, and processor(s) 201 may execute the software. Memory 203 may read the software from one or more other computer-readable media (such as mass storage device(s) 235, 236) or from one or more other sources through a suitable interface, such as network interface 220. The software may cause processor(s) 201 to carry out one or more processes or one or more steps of one or more processes described or illustrated herein. Carrying out such processes or steps may include defining data structures stored in memory 203 and modifying the data structures as directed by the software.

The memory 203 may include various components (e.g., machine readable media) including, but not limited to, a random access memory component (e.g., RAM 204) (e.g., static RAM (SRAM), dynamic RAM (DRAM), ferroelectric random access memory (FRAM), phase-change random access memory (PRAM), etc.), a read-only memory component (e.g., ROM 205), and any combinations thereof. ROM 205 may act to communicate data and instructions unidirectionally to processor(s) 201, and RAM 204 may act to communicate data and instructions bidirectionally with processor(s) 201. ROM 205 and RAM 204 may include any suitable tangible computer-readable media described below. In one example, a basic input/output system 206 (BIOS), including basic routines that help to transfer information between elements within computer system 200, such as during start-up, may be stored in the memory 203.

Fixed storage 208 is connected bidirectionally to processor(s) 201, optionally through storage control unit 207. Fixed storage 208 provides additional data storage capacity and may also include any suitable tangible computer-readable media described herein. Storage 208 may be used to store operating system 209, executable(s) 210, data 211, applications 212 (application programs), and the like.

Storage 208 can also include an optical disk drive, a solid-state memory device (e.g., flash-based systems), or a combination of any of the above. Information in storage 208 may, in appropriate cases, be incorporated as virtual memory in memory 203.

In one example, storage device(s) 235 may be removably interfaced with computer system 200 (e.g., via an external port connector (not shown)) via a storage device interface 225. Particularly, storage device(s) 235 and an associated machine-readable medium may provide non-volatile and/or volatile storage of machine-readable instructions, data structures, program modules, and/or other data for the computer system 200. In one example, software may reside, completely or partially, within a machine-readable medium on storage device(s) 235. In another example, software may reside, completely or partially, within processor(s) 201.

Bus 240 connects a wide variety of subsystems. Herein, reference to a bus may encompass one or more digital signal lines serving a common function, where appropriate. Bus 240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 200 may also include an input device 233. In one example, a user of computer system 200 may enter commands and/or other information into computer system 200 via input device(s) 233. Examples of an input device(s) 233 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. The input device is a Kinect, Leap Motion, or the like. Input device(s) 233 may be interfaced to bus 240 via any of a variety of input interfaces 223 (e.g., input interface 223) including, but not limited to, serial, parallel, game port, USB, FIREWIRE, THUNDERBOLT, or any combination of the above.

In particular embodiments, when computer system 200 is connected to network 230, computer system 200 may communicate with other devices, specifically mobile devices and enterprise systems, distributed computing systems, cloud storage systems, cloud computing systems, and the like, connected to network 230. Communications to and from computer system 200 may be sent through network interface 220. For example, network interface 220 may receive incoming communications (such as requests or responses from other devices) in the form of one or more packets (such as Internet Protocol (IP) packets) from network 230, and computer system 200 may store the incoming communications in memory 203 for processing. Computer system 200 may similarly store outgoing communications (such as requests or responses to other devices) in the form of one or more packets in memory 203 and communicated to network 230 from network interface 220. Processor(s) 201 may access these communication packets stored in memory 203 for processing.

Examples of the network interface 220 include, but are not limited to, a network interface card, a modem, and any combination thereof. Examples of a network 230 or network segment 230 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 230, may employ a wired and/or a wireless mode of communication. In general, any network topology may be used.

Information and data can be displayed through a display 232. Examples of a display 232 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 232 can interface to the processor(s) 201, memory 203, and fixed storage 208, as well as other devices, such as input device(s) 233, via the bus 240. The display 232 is linked to the bus 240 via a video interface 222, and transport of data between the display 232 and the bus 240 can be controlled via the graphics control 221. The display is a video projector. The display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 232, computer system 200 may include one or more other peripheral output devices 234 including, but not limited to, an audio speaker, a printer, a storage device, and any combinations thereof. Such peripheral output devices may be connected to the bus 240 via an output interface 224. Examples of an output interface 224 include, but are not limited to, a serial port, a parallel connection, a USB port, a FIREWIRE port, a THUNDERBOLT port, and any combinations thereof.

In addition, or as an alternative, computer system 200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

The computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. The operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Sony® PS5®, Microsoft® Xbox 360®, Microsoft® Xbox One, Microsoft® Xbox Series X, Microsoft® Xbox Series S, Nintendo® Wii®, Nintendo® Wii U®, Nintendo® Switch™, and Ouya®.

Another aspect of the disclosure herein describes a non-transitory, computer-readable medium comprising executable instructions, wherein when a processor, when executing the executable instructions, performs a method as described herein.

Data augmentation

In some embodiments, augmented data may be used to train a machine learning model such as those disclosed herein. Data augmentation is a method of using real world data to generate new samples with variations imposed on them. Such data may be beneficial to a machine learning model as augmentations are often chosen such that they will reduce confounding data present in highly complex data types such as text, or images. Data augmentation may include one of synonym replacement, random insertion, random deletion, character-level modifications, leveraging pre-trained language models for contextual augmentations (such as a BERT model), or any combination thereof. Data augmentation may include techniques for image augmentations such as cropping, rotating, grayscale, contrast scaling, brightness changes, down sampling, up sampling, blurring, or any combination thereof. Augmented data may be any of the datatypes disclosed herein.

Synthetic data

In some embodiments, synthetic data may be used to train a machine learning model. Synthetic data may comprise using algorithmic methods to produce samples that mimic real world data. Synthetic samples may include samples produced through a machine learning method such as a VAE, GAN, LLM, diffusion model or any combination thereof. Synthetic data may be produced through a rules engine. Synthetic data may be produced through entity cloning, Synthetic data may be produced through data masking. Synthetic data may be any of the datatypes disclosed herein.

### Machine Learning

This disclosure provides machine learning models as well as methods of developing the models and methods of using the models.

Machine learning models may a training phase and an inference phase. During the training phase the model, or a portion thereof, may be free to learn. During inference the model does no learn and instead produces an output based on its input(s). When a model is used that has already been trained it may be said to be pretrained. The term “pretrained” makes no assumption about the performance of the model only that it has undergone some training or utilizes parameters that have been trained in full or in part. Multiple rounds of training may be performed, when a pretrained model goes through a subsequent round of training it is often to update the model through a method such as continuous learning, fine tuning, transfer learning or other methods.

Data may be input to the model during training or inference. Data may be in many different types, but common examples include text, image, waveform, audio, tabular, vector encoding, or noise. Some models may be configured to take multiple inputs or to give multiple outputs. Some models may take multiple inputs of multiple classes of data, for example a model which takes 3 inputs; input 1 being text, input 2 being an image, and input 3 being noise. Data may be split into different sets. The first set may be a training set that is used during the training phase as input to the model. Optionally, 2 other sets may be made, a validation set which is not used to train the model but is used during training time to provide an indication of the models performance on previously untrained data, and a test set meant to be used after training is complete to test the trained model at inference time. Alternatively, a model, or a portion thereof, may take as input random noise. Such as the case with diffusion and generative adversarial models.

A machine learning model such as those disclosed here may be comprised of parameters (such as weights and biases), one or more processing steps, one or more outputs and one or more inputs. During training the machine learning model may calculate a loss useful for calculating the error between the real output of the model and the expected output of the model. Some set of the model parameters may be updated based at least in part on the loss calculation. The model may perform multiple rounds, or epochs, of training wherein an input or set of inputs is given and processed by the model which then produces an output or set of outputs which may then the basis for updating the weights. The new weights may be used in the next epoch. Some embodiments may comprise more steps. Training may occur in different environments such as supervised, unsupervised, semi-supervised, self-supervised or some combination thereof.

In a supervised environment the expected output is provided for each input during training. The training data will have labels associated with each sample of the training data. The labels are an indication of the desired output of the model when the corresponding input is given.

In an unsupervised method the training set does not have corresponding labels. The input is the desired output of the model and is used in place of a label. The desired output is communicated through a score which may be related to some other output indication. For example, in a diffusion model a particular output may not be desired, as diffusion models are often valued for their variability in their output, and the desired output is not concrete. In this example, the model training may use other information such as text descriptors as the label. In this example the model may output an image which cannot be directly compared to a text description, so a classifier of the output image may be used, and a score calculated between the classifier and the tokenized or vectorized text description. This example illustrates that training may be quite flexible and desired output is most relevant in reference to a score that is used to adjust the model's parameter through a process called backpropagation, described in more detail below.

Self-supervised training may not use labels. Unlike unsupervised learning, self-supervised methods (also called self-supervised learning or SSL) generate implicit labels from the unstructured data. In SSL, tasks fall into two categories: pretext tasks and downstream tasks. In a pretext task, SSL is used to train an AI system to learn meaningful representations of unstructured data. Those learned representations can be subsequently used as input to a downstream task, like a supervised learning task or reinforcement learning task. The reuse of a pre-trained model on a new task is referred to as “transfer learning.”

Self-supervised learning may be used in the training of a diverse array of sophisticated deep learning architectures for a variety of tasks, from transformer-based large language models (LLMs) like BERT and GPT to image synthesis models like variational autoencoders (VAEs) and generative adversarial networks (GANs) to computer vision models like SimCLR and Momentum Contrast (MoCo).

Semi supervised combines unsupervised and supervised tasks by using labeled and unlabeled data. In some cases, there may be datasets where some samples are labeled, and others are not. In this case it may be desirable to have a fully labeled dataset but producing labels for large datasets is time consuming and expensive. Semi supervised learning first trains on the labeled data of the set of data. It is then used to produce pseudo-labels, or labels that are not validated, as an inference pass. The pseudo-labels may be in a continuous range, for example 0 to 1, where the values closer to 1 are higher confidences in the pseudo-label. The pseudo-labeled data above a certain confidence threshold may be used along with the labeled data to retrain the model to improve the overall performance of the model.

A machine learning model may be trained as a classifier or regression model. In both cases a model is trained as described above. A classifier is trained to give a value corresponding to a given class or set of classes. When one class is used the model is said to be a binary classifier. As an example, a binary classifier may give a single value between 1 and 0 where 1 indicates the presence of the desired class in the input and 0 indicates the absence of the desired class in the input. A value between 0 and 1 may indicate a probability of the desired class. In some cases, the output value of a model may be compared against a threshold value, when the output is below the threshold the classifier outputs an indication that the desired class is not present, when the output is above the classifier outputs an indication that the desired class is present.

A classifier may also perform multiclass classification where more than one class is indicated in a binary fashion. In this case the model will output a single class as present and all other classes with be absent.

A classifier may be a multiclass multilabel, where more than one class may be output as present at one time. This may be useful in settings where classes may co-exist in the input. For example, an image segmentation model or object detection model may indicate the presence of multiple objects in an image and output an indication in its output for each of the detected objects. This may also be useful when the model is used to detect either multiple classes in the input and/or where some other label is desired such as a contextual output.

A regression model may be used in a predictive fashion, whereas a classifier is used to place input or portions of input into classes that are predefined. Regression models may take an input and output a continuous value as a prediction or forecast score. As an example, a regression model may take an image and predict a desired set of values describing a shape of a new object to be placed in the image. In this example the output, or a portion of the output, of a regression may be used as an input to another model.

Once training is completed, a model may be used to infer on a set of inputs. The model output may be the desired output for the use of the model or there may be some portion of the model that is used for a desired output different than the output that was used during training time. At inference time the model's weights may be static, whereas during training they may be dynamic as discussed previously.

A model may be trained again after first being trained. Such cases may include transfer learning applications, fine-tuning of the model, integration of the model into a larger model, continuous training, or some combination thereof. During fine-tuning a trained model may be trained on a different set of data, a subset of the original data or some combination thereof to cause the model to improve its performance on a given task or subtask to that on which it was previously trained. In retraining, a set of model parameters may be untrainable often so that the previously learned information represented by those parameters is maintained through the retraining process. During transfer learning a model may be trained to improve performance on a task similar to the task the model was previously trained on, for example; a model may be trained to detect a first set of objects (e.g., common objects in a set of pictures comprising images of the outdoors). The model in this example may then be trained to detect similar descriptions across clinical trial descriptions, exclusions and/or inclusions and clinical data that may not have been present in the first set of objects. For example, a model may be desired that is capable of producing a latent vector representation of text for clinical trial data such as descriptions, inclusions and/or exclusions, however the volume of data that may be used for training such a model may be insufficient for the model to achieve good generalization after training. A model pretrained on general text may have information pertinent to the task of understanding clinical trial descriptions, inclusions and/or exclusions but may not have been trained on such data, or it may be desired to improve the performance of the model on such a task. An additional round(s) of training where the training dataset may include primarily clinical trial descriptions, inclusions and/or exclusions may improve the model's performance for such a case.

A model may be integrated into another model. In those cases, a model may be trained and appended to another model that may or may not be trained. The new model, which includes the previously trained original model, may be trained such that the original model's parameters are static (the original model is in inference mode), while the rest of the new model may be trainable, or partially trainable. Such an example may seem like a transfer learning scenario, and it may be used that way, but it may also be used to connect two models via the trained model using it as a stable intermodal processor. The method may also be used to convert an input into a format that is easier for the new model to process. As such different tasks of a model's behavior may be trained independent of each other and then appended and trained in an end-to end fashion allowing the new model to benefit from the improved training regime of the independent training and to require potentially less data or time to train that might have before.

Neural networks are a class of machine learning models which use artificial neurons an individual processing units. These artificial neurons may comprise an input, a set of weights, a set of biases, a summation step and an activation function. When an artificial neuron receives an input, which may be one or more values, the input may have a weight, or a set of weights, and a bias or set of biases applied to it. The transformed input may have a function applied that combines them into one value, such as summation, in the case that multiple inputs are given to an artificial neuron, and/or processed by an activation function which outputs a value.

Multiple artificial neurons may be used to create a layer of neurons that takes in the same input and outputs a number of values equal to the number of neurons in that layer. A neural network may be composed of multiple layers. Layers may take as input data, or output from other layers or some other values such as a random value. Layers may be smaller, larger or the same size as the input they take. Layers may be of various types such as, but not limited to, the following layer types; dense, convolutional, pooling, recurrent, preprocessing, normalization, regularization, attention, reshaping, merging, or activation. When a layer's output is received as input by another layer the two layers are connected, layers may be connected to any layer that follows.

Layer connectivity may define the model's architecture. Choice of a model's architecture may be directed by the task being carried out by the layer or set of layers. Layer architectures may then be described by their function. Some examples of architectures are, feed-forward networks, recurrent neural networks (RNN), long short-term memory (LSTM), echo networks, diffusion models, transformers, visual geometry group (VGG), graph neural networks (GNN), encoders, variational autoencoders (VAE), UNET, and generative adversarial networks. Networks are generally agnostic to the layer types used in them and may comprise multiple layer types. As an example, a convolutional neural network (CNN) may be a feed forward network comprising convolutional layers as well as pooling, and flattening layers, this is only an example though and CNNS may have different architectures or layer compositions. Architectures may also be combined in one model as is the case in complex models such as diffusion models and large language models (LLM).

Object detection involves localizing and/or classifying objects within an input such as an image. It may identify specific objects and may output bounding boxes for identified objects. Object detection may comprise components for generating potential object proposals, feature extraction networks for analyzing proposals, and object classification networks for assigning class labels. Examples of object detection networks may include, but are not limited to, Faster R-CNN, YOLO (You Only Look Once), and SSD (Single Shot MultiBox Detector).

Segmentation networks may partition an input, such as an image, into meaningful regions to identify and differentiate objects or regions of interest. Segmentation networks may be used for tasks such as understanding object boundaries and extracting fine-grained information. Segmentation techniques may include semantic segmentation, which may assign class labels to each pixel, and instance segmentation, which may identify individual instances of objects. Panoptic segmentation may combine semantic and instance segmentation where it may label all pixels while distinguishing different instances.

### Recommender Systems

A recommender system may provide suggestions for items that are most pertinent to a particular user. Typically, the suggestions refer to various decision-making processes, such as what product to purchase, what music to listen to, or what online news to read. These systems can operate using a single type of input, like music, or multiple inputs within and across platforms like news, books and search queries.

Recommender systems are particularly useful when an individual needs to choose an item from a potentially overwhelming number of items that a service may offer. Disclosed herein is a recommender system that may be communicatively coupled to a database which may gather clinical trial data. The recommender may be configured to take multiple streams of data to provide a recommendation of clinical trials and treatment centers to a user (i.e., a subject or health care provider, such as a physician). In some cases, the user may not be otherwise aware of the available clinical trials or treatment centers offering those clinical trials.

### Collaborative Filtering

Collaborative filtering assumes that when two data points agreed in the past they will agree in the future. The system generates recommendations using only information about rating profiles for different users or items. By locating peer users/items with a rating history similar to the current user or item, they generate recommendations using this neighborhood. Collaborative filtering methods are classified as memory-based and model-based. An example of memory-based approaches is the user-based algorithm, while that of model-based approaches is matrix factorization.

A collaborative filtering approach may not rely on machine analyzable content and therefore may be capable of accurately recommending complex items such as movies without requiring an “understanding” of the item itself. Algorithms may be used in measuring user similarity or item similarity in recommender systems. For example, the k-nearest neighbor (k-NN) approach, and the Pearson Correlation, cosign similarity, hamming distance, or similarity scores derived from a machine learning model such as a random forest, support vector machine, logistic regression, or neural network (for example).

### Content-based Filtering

Content-based filtering methods are based on a description of the item and a profile of the user's preferences. These methods are best suited to situations where there is known data on an item (for example: name, location, description, etc.), but not on the user. Content-based recommenders treat recommendation as a user-specific classification problem and learn a classifier for the user's likes and dislikes based on an item's features.

In this system, keywords are used to describe the items, and a user profile is built to indicate the type of item this user interacts with. In other words, these algorithms try to recommend items similar to those that a user interacted with in the past or is examining in the present. It does not rely on a user sign-in mechanism and may generate this often temporary profile. In particular, various candidate items are compared with items previously interacted with by the user, and the best-matching items are recommended.

To create a user profile, the system mostly focuses on two types of information: 1. a model of the user's preference and 2. a history of the user's interaction with the recommender system.

These methods use an item profile (i.e., a set of discrete attributes and features) characterizing the item within the system. To abstract the features of the items in the system, an item presentation algorithm is applied, such as, but not limited to, term frequency and reciprocal document frequency (tf-idf) representation (vector space representation). The system creates a content-based profile of users based on a weighted vector of item features. The weights denote the importance of each feature to the user and can be computed from individually rated content vectors using a variety of techniques. Simple approaches use the average values of the rated item vector while others use machine learning techniques such as, but not limited to, Bayesian Classifiers, cluster analysis, decision trees, and artificial neural networks in order to estimate the probability that the user is going to like the item.

Recommender systems may utilize techniques such as, but not limited to text mining, information retrieval, sentiment analysis, multimodal sentiment analysis, and deep learning.

### Hybrid Recommendations Approaches

The hybrid approach combines collaborative filtering, content-based filtering, and other approaches. Examples of hybrid approaches include, but are not limited to, making content-based and collaborative-based predictions separately and then combining them. by adding content-based capabilities to a collaborative-based approach (and vice versa); by unifying the approaches into one model, for example Some hybridization techniques include:
Weighting: Combining the score of different recommendation components numerically.
Switching: Choosing among recommendation components and applying the selected one.
Mixing: Recommendations from different recommenders are presented together to give the recommendation.
Feature Combination: Features derived from different knowledge sources are combined and given to a single recommendation algorithm.
Feature Augmentation: Computing a feature or set of features, which is then part of the input to the next technique.
Cascade: Recommenders are given strict priority, with the lower priority ones breaking ties in the scoring of the higher ones.
Meta-level: One recommendation technique is applied and produces some sort of model, which is then the input used by the next technique.

### Web Application

A computer program includes a web application. In light of the disclosure provided herein, those of skill in the art will recognize that a web application, in various embodiments, utilizes one or more software frameworks and one or more database systems. A web application is created upon a software framework such as Microsoft®.NET, python, java JavaScript, NodeJS, JavaScript react, Ruby on Rails (RoR). A web application utilizes one or more database systems including, by way of non-limiting examples, relational, non-relational, object oriented, associative, XML, and document-oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. A web application is written to some extent in a markup language such as Hypertext Markup Language (HTML), Extensible Hypertext Markup Language (XHTML), or extensible Markup Language (XML). A web application is written to some extent in a presentation definition language such as Cascading Style Sheets (CSS). A web application is written to some extent in a client-side scripting language such as Asynchronous JavaScript and XML (AJAX), Flash® ActionScript, JavaScript, or Silverlight®. A web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. A web application is written to some extent in a database query language such as Structured Query Language (SQL). A web application integrates enterprise server products such as IBM® Lotus Domino®. A web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

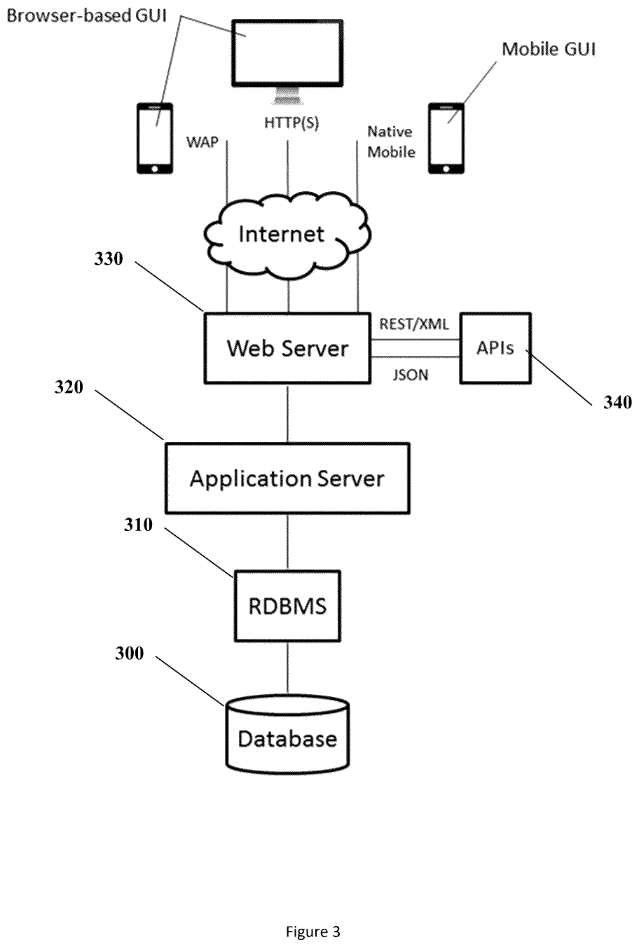

Referring to FIG. 3, in a particular embodiment, an application provision system may include one or more databases 300 accessed by a relational database management system (RDBMS) 310. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system may include one or more application severs 320 (such as Java servers, NodeJS, ExpressJS, .NET servers, PHP servers, and the like) and one or more web servers 330 (such as Apache, IIS, GWS and the like). The web server(s) optionally expose one or more web services via app application programming interfaces (APIs) 440. Via a network, such as the Internet, the system provides browser-based and/or mobile native user interfaces.

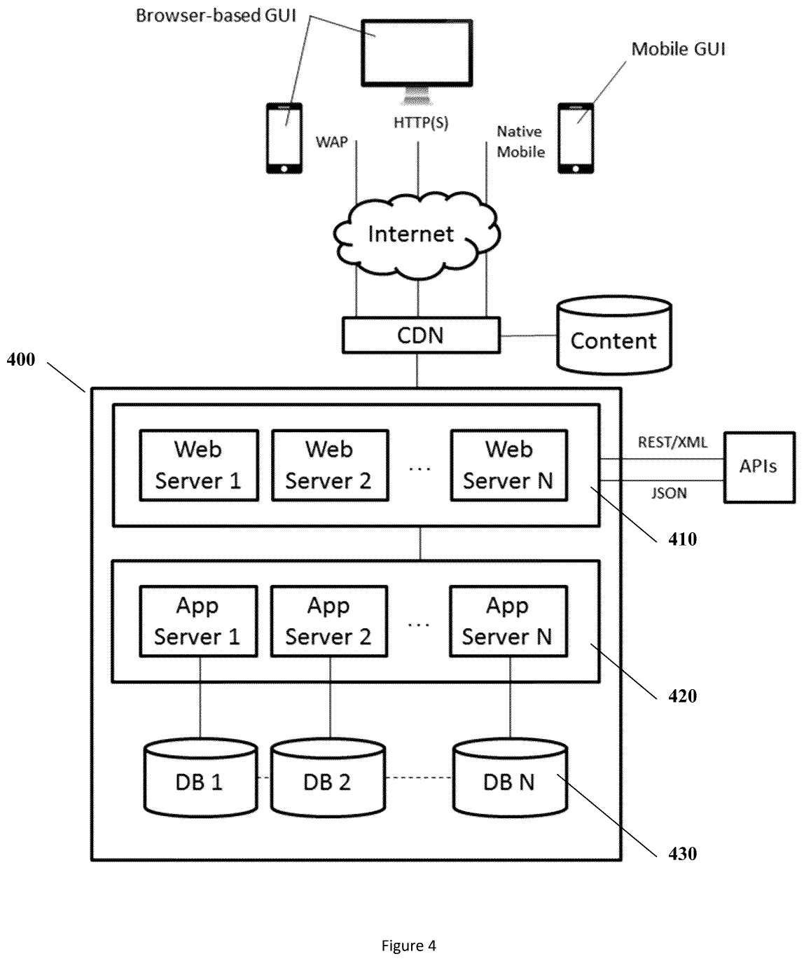

Referring to FIG. 4, in a particular embodiment, an application provision system alternatively has a distributed, cloud-based architecture 400 and may include elastically load balanced, auto-scaling web server resources 410 and application server resources 420 as well synchronously replicated databases 430.

### Mobile Application

A computer program includes a mobile application provided to a mobile computing device. The mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, Rails, VB.NET, WML, and XHTML/HTML with or without CSS, React native, react, swift, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. A computer program includes one or more executable complied applications.

### Web Browser Plug-in

The computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may include one or more web browser extensions, add-ins, or add-ons. The toolbar may include one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer, Mozilla® Firefox®, Google® Chrome, Apple® Safari®, Opera Software® Opera®, and KDE Konqueror. The web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module may include a file, a section of code, a programming object, a programming structure, a distributed computing resource, a cloud computing resource, or combinations thereof. In further various embodiments, a software module may include a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

The platforms, systems, media, and methods disclosed herein include one or more databases, or use of the same. In view of the disclosure provided herein, those of skill in the art will recognize that many databases are suitable for storage and retrieval of data such as, but not limited to, clinical trial descriptions, inclusions and/or exclusions, clinical data, or any combination thereof. In various embodiments, suitable databases include, by way of non-limiting examples, relational databases, non-relational databases, object-oriented databases, object databases, entity-relationship model databases, associative databases, XML databases, document-oriented databases, and graph databases. Further non-limiting examples include SQL, PostgreSQL, MySQL, Oracle, DB2, Sybase, and MongoDB. A database is Internet-based. In further embodiments, a database is web-based. In still further embodiments, a database is cloud computing based. In a particular embodiment, a database is a distributed database. In other embodiments, a database is based on one or more local computer storage devices.

### Data Transmission

The subject matter described herein, including methods and systems as described herein and may be configured to be performed in one or more facilities at one or more locations. Facility locations are not limited by country and include any country or territory. In some instances, one or more steps are performed in a different country than another step of the method. In some embodiments, one or more method steps involving a computer system are performed in a different country than another step of the methods provided herein. In some embodiments, data processing and storage are performed in a different country or location than one or more steps of the methods described herein. In some embodiments, one or more products or data are transferred from one or more of the facilities to one or more different facilities for analysis or further analysis. Data includes, but is not limited to, information regarding the stratification of a subject, and any data produced by the methods disclosed herein. In some embodiments of the methods and systems described herein, the subject information is compiled, and a subsequent data transmission step will transmit or store the subject information.

Any step of any method described herein may be performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as queries, objects, properties, types, filters, tables, or any combination thereof, performed in one facility in a particular location and corresponding data transmitted to another location.

### Business Methods Utilizing a Computer

The methods described herein may utilize one or more computers. The computer may be used for managing customer and subject information. The computer may include a monitor or other user interface for displaying data, results, billing information, marketing information (e.g., demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party.

The entity entering or reviewing information into a database for the purpose of one or more of the following: inventory tracking, order tracking, customer management, customer service, billing, and sales. Sample information may include, but is not limited to customer name, unique customer identification, or any information suitable for storage in a database.

The database may be accessible by a user. Database access may take the form of electronic communication such as a computer or telephone. The database may be accessed through an intermediary such as a customer service representative, business representative, or consultant. The availability or degree of database access may change upon payment of a fee for products and services rendered or to be rendered.

### TERMINOLOGY

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusions of such definitions herein should not necessarily be construed to represent a substantial difference over what is generally understood in the art.

“Physician” refers generally to any health care provider including but not limited to a primary care physician, a medical specialist, a neurologist, a radiologist, a geneticist, and a medical assistant, among others.

“Clinical trial” refers to a research study in which human volunteers are assigned to interventions (e.g., a medical product, behavior, or procedure) based on a protocol and are then evaluated for effects on biomedical or health outcomes.

### Examples

The following examples are included for illustrative purposes only and are not intended to limit the scope of the inventive concepts.

### Example 1: Matching of a Client to a Treatment Center and Clinical Trial

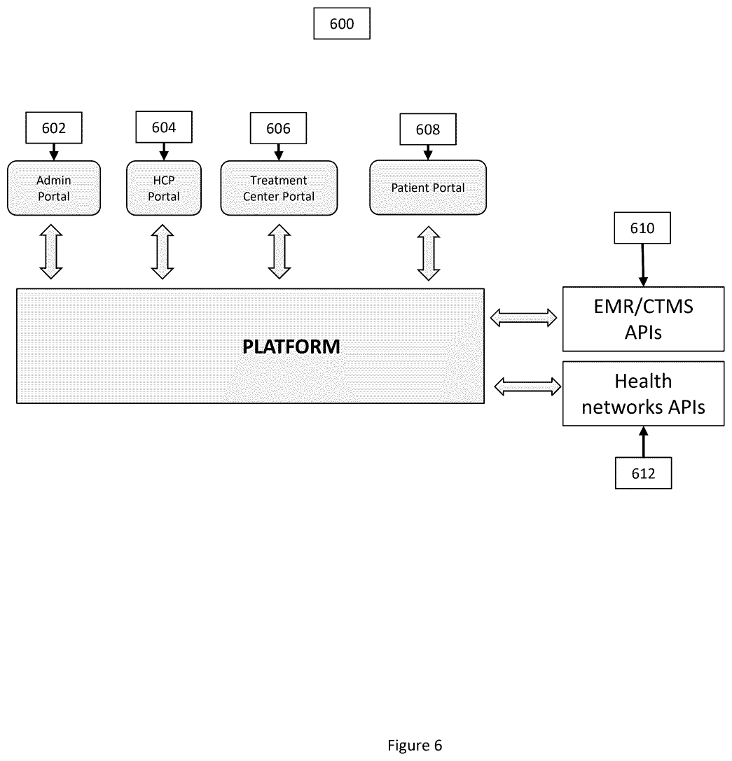

A user, after navigating to the appropriate associated with the methods disclosed herein, may access a platform 600, such as those illustrated in FIG. 6. The portal being access may be limited based upon the type of user. A user may be a healthcare provider (HPC) and would be routed to the HPC portal 604. A user may be a subject and is routed to the subject portal 608. A user may be a member of a treatment center team and is routed to the treatment center portal 606. A user may be an administrator and is routed to the admin portal 602.

The platform 600 may interact with an API associated with electronic medical record (EMR) or clinical trial management system (CTMS) 610. A CTMS may provide contact management, calendar and monitoring services, document management, a project plan (i.e., study milestones and task progress tracking), contracting and payment systems, subject tracking and electronic data capture integration, visit report authorization system, letter generation, business analytics and reporting.

The platform may interact with a health network API 612. This API may provide subject insurance profiles, claims submission system for HCPs and institutions, a claims status system, claim response and reporting, claim attachment submission, application status monitoring, attachment retrieval, institutional and/or professional integrated rules, and payment services.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A computer-implemented method to match subjects with a treatment center comprising, via a computer system, comprising:
a. receiving a set of clinical data of a subject from a user;
b. processing the set of data based at least in part on a computer program configured to identify and output a set of information, wherein the set of information is related to a treatment;
c. identifying, through an algorithm, a list of treatment centers based at least in part on the set of information; and
d. providing an output identifying relevant treatment centers.

64. The method of claim 63, wherein the set of data comprises at least one of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, a measure of subject prognosis, or any combination thereof.

65. The method of claim 64, wherein the clinical code comprises at least one of an ICD10 code, an ICD-11 code, a SNOMED CT code, a LOINC code, a CPT code, a HCPCS code, an ICD-O code, a DRG code, a Read Code, an RxNorm code, an ATC code, or any combination thereof.

66. The method of claims 63, wherein the clinical data comprise HIPAA-limited data.

67. The method of claim 63, wherein the algorithm comprises a rule-based system.

68. The method of claim 67, wherein the rule-based system parses at least an FDA approved indication of clinical trial.

69. The method of claim 67, wherein the rule-based system comprises:
a. a rule for minimizing distance between a subject and a treatment center; and
b. a rule for matching a set of treatment centers with the subject.

70. The method of claim 69, wherein the rule of (a) is based on a distance threshold.

71. The method of claim 70, wherein the distance threshold is tunable.

72. The method of claim 69, wherein the set of treatment centers houses a clinical trial that matches with at least a portion of the set of information relevant to a treatment.

73. The method of claim 72, wherein the clinical trial comprises at least one of a cellular therapy clinical trial, a gene therapy clinical trial, a radioligand clinical trial, a tissue engineered product clinical trial, a somatic cell therapy medicinal product, or any combination thereof.

74. The method of claim 63, wherein the computer program processing comprises a large language model.

75. The method of claim 63, wherein the identifying comprises:
a. parsing, through the algorithm, an FDA-approved indication associated with a treatment;
b. associating the set of information with the parsed FDA-approved indication; and
c. pulling the list of treatment centers where the treatment is performed.

76. The method of claim 63, wherein the algorithm comprises a machine learning model.

77. The method of claim 76, wherein the machine learning model comprises at least one of an autoencoder, a long short-term memory model, a large language model, a recurrent neural network, a clustering algorithm, a transformer, or any combination thereof.

78. The method of claim 63, wherein the set of data comprises a plurality of members selected from the group consisting of subject demographic data, diagnosis data, clinical code, referring health care provider data, referral location, date of referral, inbound treatment center contact information, subject history data, familial history data, medical history data, lab result data, health survey data, ICEES data, COHD data, HuSH data, HuSH+ clinical data, treatment center capacity, treatment center turn-around time, treatment center operational data, and a measure of subject prognosis.

79. A method for optimizing workflows, comprising:
a. providing an orchestration hub comprising a shared context data store, an intake agent, a billing agent, and a meta-agent where the intake agent comprising a set of intake agent parameters, billing agent comprising a set of billing agent parameters and meta agent;
b. routing by the orchestration hub a series of tasks through the intake agent and/or the billing agent according to a learned routing policy to produce a set of outcome data;
c. collecting the set of outcome data for the intake agent and billing agent;
d. assessing the outcome data by the meta-agent;
e. altering the intake agent parameters, or the billing agent parameters or both to optimize the outcome data; and
f. providing an optimized intake agent, or billing agent or both.

80. The method of claim 79, further comprising providing a document intelligence agent.

81. The method of claim 80, wherein the document intelligence agent comprises a vision language transformer configured to extract structured entities from image based documents.

82. The method of claim 79, wherein the routing policy is learned through a reinforcement learning method.
